(19) United States
(12) Patent Application Publication
(10) Pub. No.: US 2005/0203407 A1
(43) Pub. Date: Sep. 15, 2005

(54) Ultrasonic diagnostic apparatus

(75) Inventor: Masatoshi Yoshihara, Saitama-shi (JP)

Correspondence Address: SNIDER & ASSOCIATES, P. O. BOX 27613, WASHINGTON, DC, 20038-7613, US

(73) Assignee: Fujinon Corporation, Saitama-shi (JP)

(21) Appl. No.: 11/065,091

(22) Filed: Feb. 25, 2005

(30) Foreign Application Priority Data
Feb. 25, 2004 (JP) 2004-048993
Feb. 25, 2004 (JP) 2004-048994

(51) Int. Cl. A61B 8/00; A61B 8/12

(52) U.S. Cl. 600/458; 600/437

(57) ABSTRACT

An ultrasonic diagnostic apparatus comprising a probe, the probe including at least one transducer that generates an ultrasonic wave, so as to form an ultrasonic tomographic image, wherein a plurality of transmission signals are generated to at least one of said at least one transducer in a common scan line period, said plurality of transmission signals including a first transmission signal and at least one second transmission signal subsequent to the first transmission signal, wherein said at least one second transmission signal is generated when a first ultrasonic wave resulting from the first transmission signal is generated, so as to form at least one second ultrasonic wave, and wherein the first ultrasonic wave and said at least one second ultrasonic wave are combined with each other, so as to form a synthesized ultrasonic wave.

### BACKGROUND OF THE INVENTION

1. Field of the Invention

The present invention relates to an ultrasonic diagnostic apparatus which forms a tomographic image or the like of a test body, and more particularly to a configuration for controlling the waveform of a generated ultrasonic wave.

2. Description of the Related Art

In an ultrasonic diagnostic apparatus, a probe having a single transducer or a plurality of transducers transmits and receives an ultrasonic wave to and from a test body such as the inside of the human body, and a reception signal obtained from the probe is processed so that a tomographic image, blood flow information, or the like of the test body is displayed on a monitor (displaying device) to be observed (for example, see JP-A-2002-034975, JP-A-2002-052025 and JP-A-2002-065671).

FIGS. 6A and 6B show a transmission signal which is supplied to a single transducer in one ultrasonic scan line, and a reception signal which is received from one reflector. When the transmission signal Sa of FIG. 6A is applied to the transducer, a generated ultrasonic wave is transmitted toward the reflector (test body). An ultrasonic wave which returns from the reflector is received by the same transducer, and the reception signal Ra of FIG. 6B is obtained. In FIG. 6B, the waveform g₁ shows leakage of the transmission signal.

FIGS. 7A and 7B shows enlarged waveforms of the transmission signal and the reception signal. As shown in FIG. 7A, the transmission signal Sa has a trigger waveform of an amplitude a₀ (about several tens to several hundreds of V) and a pulse width t₀. As shown in FIG. 7B, the reception signal Ra has a waveform in which the amplitude (maximum amplitude A₀) is varied, and which continues for the ultrasonic wave (generation) period H₀ of, for example, 5 to 6 cycles. In the reception signal Ra, as the ultrasonic wave period H₀ is shorter, the distance resolution is higher, and, as the amplitude (wave height) is larger, the sensitivity is higher.

In an ultrasonic diagnostic apparatus, it is requested to further improve the quality of an ultrasonic image. When the above-mentioned distance resolution and search sensitivity in ultrasonic transmitted and received waves are enhanced more than those in the related art, it is possible to obtain an image which has a high image quality, and which can be easily observed.

### SUMMARY OF THE INVENTION

The invention has been conducted in view of the above-discussed problems. It is an object of the invention to provide an ultrasonic diagnostic apparatus in which the distance resolution and the search sensitivity can be enhanced, and which can obtain an image that has a high image quality, and that is easily observed.

(1) In order to attain the object, there is provided an ultrasonic diagnostic apparatus comprising a probe, the probe including at least one transducer that generates an ultrasonic wave, so as to form an ultrasonic tomographic image, wherein a plurality of transmission signals are generated to at least one of said at least one transducer in a common scan line period, said plurality of transmission signals including a first transmission signal and at least one second transmission signal subsequent to the first transmission signal, wherein said at least one second transmission signal is generated when a first ultrasonic wave resulting from the first transmission signal is generated, so as to form at least one second ultrasonic wave, and wherein the first ultrasonic wave and said at least one second ultrasonic wave are combined with each other, so as to form a synthesized ultrasonic wave having a controlled waveform.

(2) There is provided the ultrasonic diagnostic apparatus as set forth in (1), wherein an initial one of said at least one second ultrasonic wave is generated with forming a shift of approximately nT/4 where n is an odd number and T is a cycle of a waveform of the first ultrasonic wave, to control a generation period of the synthesized ultrasonic wave to be shortened as compared with that of the first ultrasonic wave.

(3) There is provided the ultrasonic diagnostic apparatus as set forth in (1), wherein adjacent ones of a plurality of ultrasonic waves are generated with forming a shift of approximately one cycle, said plurality of ultrasonic waves resulted from said plurality of transmission signals, so as to control an amplitude of the synthesized ultrasonic wave to be increased as compared with that of the first ultrasonic wave.

According to the configuration of the invention, an ultrasonic wave which is to be transmitted and received in one ultrasonic scan line (one direction) is formed by plural transmission (pulse) signals. When the timings of outputting the transmission signals are adjusted, or the delay amount for the second and subsequent ultrasonic waves with respect to the first ultrasonic wave is controlled, the resulting synthesized ultrasonic waveform can be arbitrarily changed. In the case of (2) above, two ultrasonic waves in which the amplitude (wave height) is made smaller than that in the related art are generated with shifting from each other by, for example, ¼ (or ¾, 5/4, or the like) cycle, and then combined with each other. As a result, the ultrasonic wave generation period is shortened, and hence the distance resolution can be enhanced. In the case of (3) above, for example, two ultrasonic waves which are shifted from each other by one cycle are combined with each other, so that the amplitude is increased. Therefore, the search sensitivity can be enhanced.

(4) There is provided the ultrasonic diagnostic apparatus as set forth in (1), wherein an amplitude of each of said plurality of transmission signals is variably adjusted.

(5) There is provided the ultrasonic diagnostic apparatus as set forth in (4), wherein a pulse width of each of said plurality of transmission signals is variably adjusted.

(6) There is provided the ultrasonic diagnostic apparatus as set forth in (1), wherein a pulse width of each of said plurality of transmission signals is variably adjusted.

According to the configuration of (4) above, an ultrasonic wave which is to be transmitted and received in one ultrasonic scan line (one direction) is formed by plural transmission (pulse) signals. When the timings of outputting the transmission signals are adjusted, or the delay amount for the second and subsequent ultrasonic waves with respect to the first ultrasonic wave is controlled, and the amplitudes of the transmission signals are adjusted respectively to different values, the resulting synthesized ultrasonic waveform can be arbitrarily changed. When the amplitudes (wave heights) of the ultrasonic waves are adjusted so as to be increased, it is possible to enhance the search sensitivity.

In the case of (6) above, when the delay amount for the second and subsequent ultrasonic waves with respect to the first ultrasonic wave is controlled, and the pulse widths of the transmission signals are adjusted respectively to different values, the resulting synthesized ultrasonic waveform can be arbitrarily changed. When both the amplitudes and pulse widths of the transmission signals are adjusted as in the case of (5) above, the ultrasonic waveform can be changed to a desired one.

(7) There is provided an ultrasonic diagnostic apparatus comprising: a probe that includes a transducer generating an ultrasonic wave; a transmitting section that outputs a plurality of transmission signals to the transducer; a receiving section that receives a reception signal from the transducer; a detection section that detects the reception signal, the detection section being connected to the receiving section; an A/D converter that analog/digital-converts an output of the detection circuit; a digital scan converter that applies scan conversion to an output of the A/D converter; a monitor that displays an ultrasonic image based on an output of the digital scan converter; and a control section connected to the transmitting section, wherein the transmitting section comprises: at least one transmitting circuit; and a delaying circuit connected to at least one of said at least one transmitting circuit and to the control section, wherein the control section sends a plurality of transmission triggers to said at least one transmitting circuit, at least one of said plurality of transmission triggers being routed through the delaying circuit, wherein each of said at least one transmitting circuit receives each of said plurality of transmission triggers in the different timing, so as to output each of said plurality of transmission signals in the different timing.

(8) There is provided the ultrasonic diagnostic apparatus as set forth in (7), wherein the control section sends, to one of said at least one transmitting circuit, one of said plurality of transmission triggers which is not being routed through the delaying circuit, and sends, to the delaying circuit, the other one of said plurality of transmission triggers.

(9) There is provided the ultrasonic diagnostic apparatus as set forth in (7), further comprising an amplitude control section that variably adjusts an amplitude of each of said plurality of transmission signals.

(10) There is provided the ultrasonic diagnostic apparatus as set forth in (7), further comprising a pulse width control section that variably adjusts a pulse width of each of said plurality of transmission signals.

### BRIEF DESCRIPTION OF THE DRAWINGS

FIG. 1 is a block diagram showing the whole configuration of an ultrasonic diagnostic apparatus of an embodiment of the invention;

FIGS. 2A and 2B are block diagrams showing two examples of the configurations of a transmitting section in the first embodiment;

FIG. 3A is a waveform chart showing a transmission signal in one ultrasonic scan line in the embodiment;

FIG. 3B is a waveform chart showing a reception signal in one ultrasonic scan line in the embodiment;

FIGS. 4A to 4F are enlarged waveform charts illustrating combining of ultrasonic waveforms for enhancing the distance resolution in the first embodiment;

FIGS. 5A to 5D are enlarged waveform charts illustrating combining of ultrasonic waveforms for enhancing the search sensitivity in the first embodiment;

FIG. 6A is a waveform chart showing a transmission signal in one ultrasonic scan line in the related art;

FIG. 6B is a waveform chart showing a reception signal in one ultrasonic scan line in the related art;

FIG. 7A is an enlarged waveform chart showing the transmission signal in FIG. 6A;

FIG. 7B is an enlarged waveform chart showing the reception signal in FIG. 6B;

FIG. 8 is a block diagram showing an example of the configuration of a transmitting section in the second embodiment;

FIG. 9 is a block diagram showing another example of the configuration of the transmitting section in the second embodiment;

FIGS. 10A and 10B are waveform charts showing examples in the case where the amplitudes of first and second transmission signals in the second embodiment are set to different values;

FIGS. 10C and 10D are waveform charts showing examples in the case where the pulse widths of first and second transmission signals in the second embodiment are set to different values;

FIGS. 11A to 11F are enlarged waveform charts illustrating combining of ultrasonic waveforms for enhancing the distance resolution in the second embodiment;

FIGS. 12A and 12B are enlarged waveform charts illustrating another example of combining of ultrasonic waveforms for enhancing the distance resolution in the second embodiment.

FIGS. 13A to 13C are waveform charts illustrating combining of ultrasonic waveforms for enhancing the search sensitivity in the second embodiment.

### DETAILED DESCRIPTION OF THE INVENTION

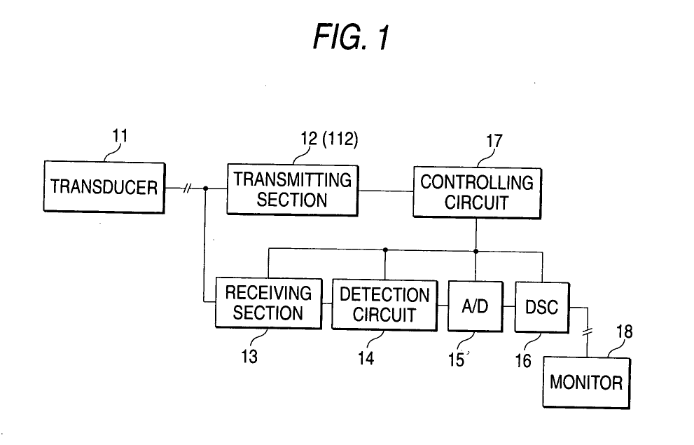

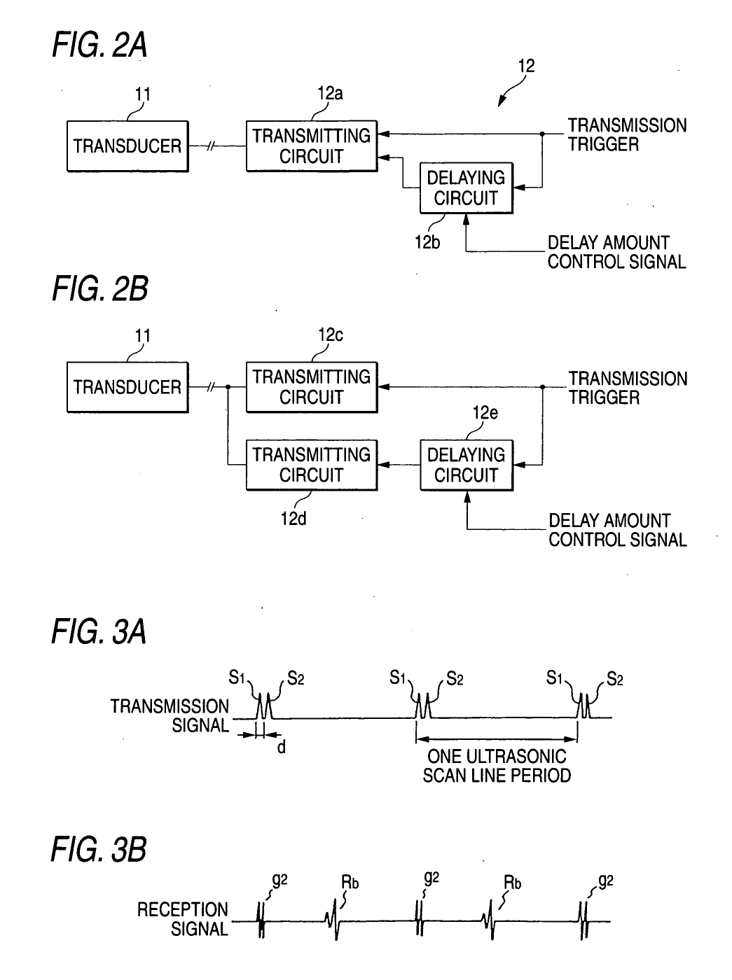

FIG. 1 shows the whole configuration of an ultrasonic diagnostic apparatus of a first embodiment, and FIGS. 2A and 2B show the configurations of a transmitting section in FIG. 1. In the ultrasonic diagnostic apparatus shown in FIG. 1, the transmitting section 12 which performs a transmission process, and a receiving section 13 which performs a reception process are connected to one or more transducers 11 disposed in a probe. A detection circuit 14 which detects a reception signal, an A/D converter 15 which analog/digital-converts an output of the detection circuit 14, and a digital scan converter (DSC) 16 which applies conversion (scan conversion) from data in a sound ray space to those in a physical space on an output of the A/D converter 15 are connected to the receiving section 13. In the apparatus, a controlling circuit 17 which controls these circuits, and a monitor 18 which displays an ultrasonic image based on an output of the DSC 16 are further disposed.

FIGS. 2A and 2B show two configurations of the transmitting section 12. FIG. 2A shows a configuration in the case where one transmitting circuit outputs a plurality of transmission signals, and FIG. 2B shows a configuration in the case where two transmitting circuits output a plurality of transmission signals. In the transmitting section 12 of FIG. 2A, one transmitting circuit 12a and a delaying circuit 12b are disposed. In accordance with a delay amount control signal supplied from the controlling circuit 17, the delaying circuit 12b sets the delay amount (time) of second and subsequent transmission (pulse) signals with respect to a first transmission (pulse) signal. Specifically, the delaying circuit 12b supplies a trigger signal which lags an incoming transmission trigger by a predetermined amount (d), to the transmitting circuit 12a. The transmitting circuit 12a first receives the transmission trigger, directly from the controlling circuit 17, and outputs the first transmission signal. On the basis of the trigger signal supplied from the delaying circuit 12b, the transmitting circuit then outputs the second transmission signal which is delayed by the predetermined amount (d).

In the transmitting section 12 of FIG. 2B, a first transmitting circuit 12c, a second transmitting circuit 12d, and a delaying circuit 12e are disposed. In this case, the first transmitting circuit 12c which receives the transmission trigger from the controlling circuit 17 outputs the first transmission signal. The delaying circuit 12e forms a trigger signal which lags from the transmission trigger by the predetermined amount (d), and the second transmitting circuit 12d which receives the trigger signal outputs the second transmission signal which is delayed by the predetermined amount (d). In the case where three or more transmission signals are to be sequentially output, the above-described transmitting circuits (12d) and delaying circuits (12e) may be further added, or plural transmission signals may be formed and output by two sets of a transmitting circuit and a delaying circuit.

In the above, the configuration of the first embodiment has been schematically described. Next, the function in the case where an ultrasonic wave is formed by two transmission signals will be described. FIGS. 3A and 3B show a transmission signal which is supplied to the single transducer, and a reception signal which is received from one reflector. In the embodiment, as shown in FIG. 3A, a first transmission signal S₁ and a second transmission signal S₂ are successively output in the same ultrasonic scan line, and the two transmission signals are given to the transducer 11. In the transducer 11, ultrasonic waves which are obtained by the respective transmission signals are combined with each other, and the resulting synthesized ultrasonic wave is transmitted to and received from a test body. The reception signal of the ultrasonic wave reflected by one reflector is obtained as shown by a reception signal R_{b} in FIG. 3B. The waveform g₂ in FIG. 3B shows leakage of the transmission signals.

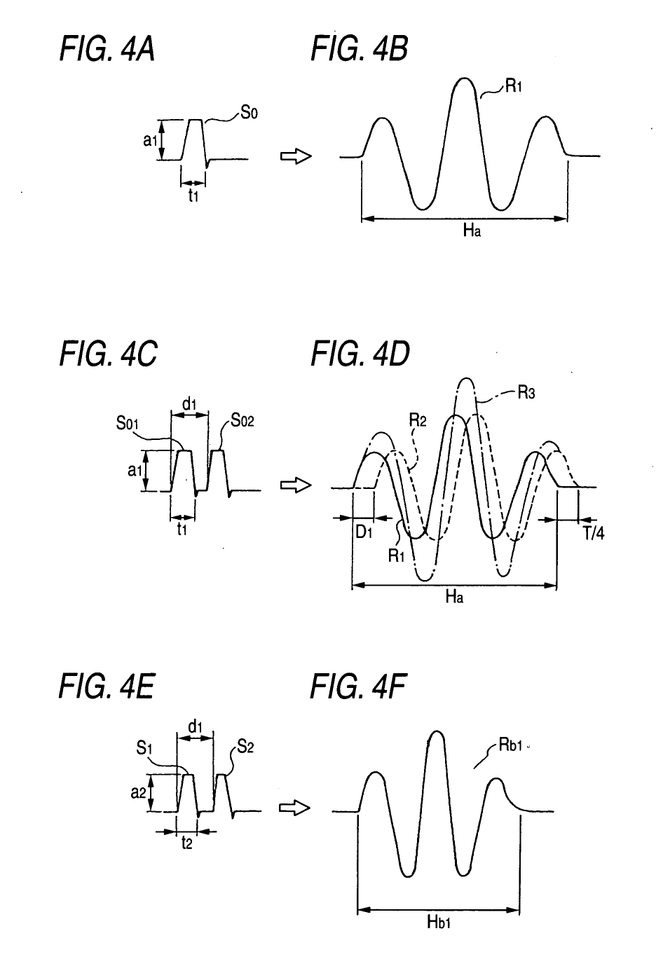

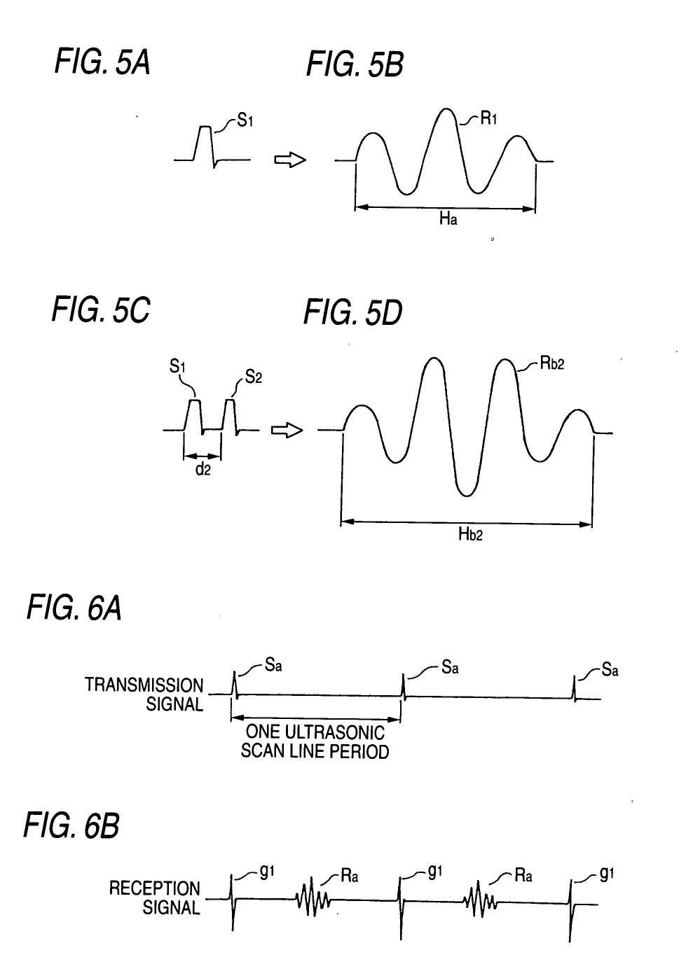

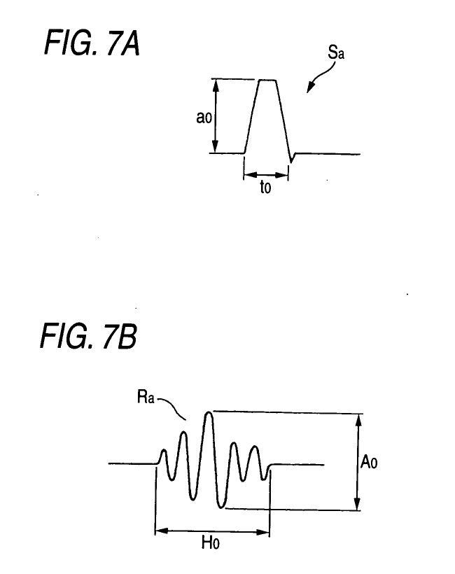

In the first embodiment, when the delay amount (time) d between the first transmission signal S₁ and the second transmission signal S₂ in FIG. 3A is variably adjusted, the distance resolution and the sensitivity can be enhanced as shown in FIGS. 4A to 4F and 5A to 5D. FIG. 4 shows waveforms in the case where the distance resolution is to be enhanced. When a transmission signal S₀ of an amplitude a₁ and a pulse width t₁ is used as shown in FIG. 4A, an ultrasonic (reception) waveform due to the signal S₀ is obtained as a waveform R₁ in an ultrasonic wave generation period Hₐ shown in FIG. 4B. When the transmission signal S₀ is used as a first transmission signal S₀₁, and also as a second transmission signal S₀₂ with forming an interval of, for example, a previously adjusted delay amount d₁ as shown in FIG. 4C, the delay amount (time) D₁ of a second ultrasonic waveform R₂ with respect to a first ultrasonic waveform R₁ which is generated by the transducer 11 is T/4 (T: cycle of the ultrasonic waveform), and an ultrasonic wave (reception wave) of a waveform R₃ which is a combination of these ultrasonic waves (waveforms R₁ and R₂) is obtained as shown in FIG. 4D. The ultrasonic waveform R₃ is longer by a period of T/4 than the ultrasonic wave generation period Hₐ, but the amplitude (wave height) is higher than that in the case where an ultrasonic wave is generated by the single transmission signal S₀.

It will be seen that, when the amplitude of the ultrasonic wave of FIG. 4D is reduced so as to be lowered to the level shown in FIG. 4B, the ultrasonic wave generation period H_{b1} becomes shorter than the period Hₐ as indicated by the ultrasonic waveform R_{b1} in FIG. 4F. In the embodiment, in order to obtain such an ultrasonic waveform, as shown in FIG. 4E, the first and second transmission signals S₁, S₂ (an amplitude a₂ and a pulse width t₂) which is smaller in amplitude (a₂<a₁) and also in pulse width (t₂<t₁) than the transmission signal S₀ of FIG. 4A are used, whereby an ultrasonic wave [FIG. 4F] of the shorter ultrasonic wave generation period H_{b1} is generated so that the distance resolution is enhanced. In FIG. 4F, while the period H_{b1} is made shorter than that in the related art, the amplitude of the ultrasonic wave may be maintained higher than that in the related art. In this case, also the search sensitivity can be enhanced. Alternatively, the delay amount between ultrasonic waves may be set to a value other than nT/4. In the alternative also, the ultrasonic wave generation period H_{b1} can be shortened.

FIGS. 5A to 5D show waveforms in the case where the search sensitivity is to be enhanced. In this case, an ultrasonic waveform R₁ of FIG. 5B which is generated by a transmission signal S₁ of FIG. 5A is composed with forming a shift of one cycle. As shown in FIG. 5C, namely, a second transmission signal S₂ is output with forming a previously adjusted delay amount d₂ with respect to the first transmission signal S₁. Therefore, two ultrasonic waveforms R₁ of FIG. 5B are combined with each other with the shift of one cycle, and an ultrasonic waveform R_{b2} having a high amplitude is obtained as shown in FIG. 5D. The ultrasonic wave generation period H_{b2} of the ultrasonic waveform R_{b2} is longer by the degree corresponding to one cycle than the period Hₐ in the case where the combination is not conducted. However, the resulting ultrasonic waveform has a larger amplitude, and hence the search sensitivity can be enhanced.

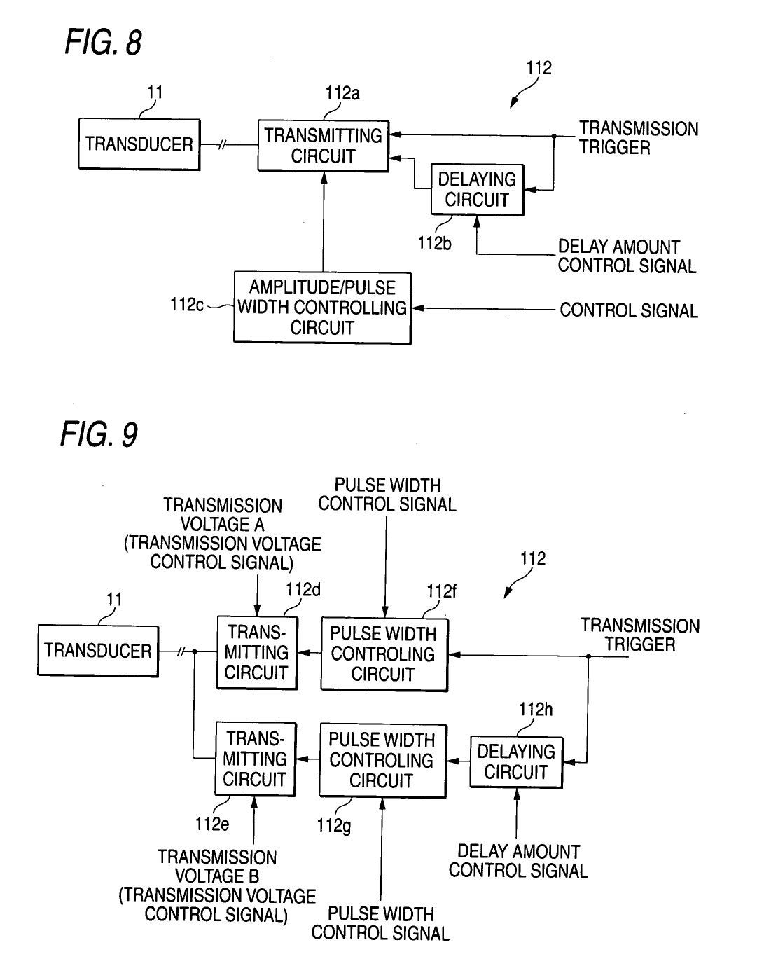

FIG. 1 shows the whole configuration of an ultrasonic diagnostic apparatus of a second embodiment, and FIGS. 8 and 9 show the configuration of a transmitting section in FIG. 1. In the ultrasonic diagnostic apparatus shown in FIG. 1, the transmitting section 112 which performs a transmission process, and a receiving section 13 which performs a reception process are connected to one or more transducers 11 disposed in a probe. A detection circuit 14 which detects a reception signal, an A/D converter 15 which analog/digital-converts an output of the detection circuit 14, and a digital scan converter (DSC) 16 which applies conversion (scan conversion) from data in a sound ray space to those in a physical space on an output of the A/D converter 15 are connected to the receiving section 13. In the apparatus, a controlling circuit 17 which controls these circuits, and a monitor 18 which displays an ultrasonic image based on an output of the DSC 16 are further disposed.

FIG. 8 shows an example of the configuration of the transmitting section 112 in the case where one transmitting circuit outputs a plurality of transmission signals. In the transmitting section 112 of FIG. 8, one transmitting circuit 112a, a delaying circuit 112b, and an amplitude/pulse width controlling circuit 112c are disposed. In accordance with a delay amount control signal supplied from the controlling circuit 17, the delaying circuit 112b sets the delay amount (time) for second and subsequent transmission (pulse) signals with respect to a first transmission (pulse) signal. The amplitude/pulse width controlling circuit 112c sets the amplitude or pulse width of each of the first and second (third, . . . ) transmission signals.

Specifically, the delaying circuit 112b supplies a trigger signal which lags an incoming transmission trigger by a predetermined amount (d), to the transmitting circuit 112a. The transmitting circuit 112a first receives the transmission trigger, directly from the controlling circuit 17, and forms the first transmission signal. On the basis of the trigger signal supplied from the delaying circuit 112b, the transmitting circuit then forms the second transmission signal which is delayed by the predetermined amount (d). At the same time, the amplitude/pulse width controlling circuit 112c is controlled on the basis of the control signal supplied from the controlling circuit 17, so that the first and second (third, . . . ) transmission signals having different amplitudes or pulse widths (one or both of the amplitude and the pulse width are controlled to different values) are output.

FIG. 9 shows another example of the configuration of the transmitting section 112 in the case where two transmitting circuits output a plurality of transmission signals. In the transmitting section 112 of FIG. 9, a first transmitting circuit 112d which receives a transmission voltage A (transmission voltage control signal) for controlling the amplitude or the like, a second transmitting circuit 112e which similarly receives a transmission voltage B (transmission voltage control signal), pulse width controlling circuits 112f, 112g which receive a pulse width control signal, and a delaying circuit 112h which is connected to one of the pulse width controlling circuits, or the controlling circuit 112g are disposed. In this case, on the basis of the transmission trigger supplied from the controlling circuit 17, the first transmitting circuit 112d and the pulse width controlling circuit 112f output a first transmission signal in which the pulse width is controlled. The delaying circuit 112h forms a trigger signal which lags the transmission trigger signal by the predetermined amount (d). On the basis of the trigger signal, the second transmitting circuit 112e and the pulse width controlling circuit 112g output a second transmission signal which lags from the first transmission signal by the predetermined amount (d), and in which the amplitude and the pulse width are controlled to different values. In the case where three or more transmission signals are to be sequentially output, the above-described transmitting circuits (112e) and delaying circuits (112h) may be further added, or plural transmission signals may be formed and output by two sets of a transmitting circuit, a pulse width controlling circuit, and a delaying circuit.

In the above, the configuration of the second embodiment has been schematically described. Next, the function in the case where an ultrasonic wave is formed by two transmission signals will be described. FIGS. 3A and 3B show a transmission signal which is supplied to the single transducer, and a reception signal which is received from one reflector. In the second embodiment, as shown in FIG. 3A, a first transmission signal S₁ and a second transmission signal S₂ are successively output in the same ultrasonic scan line, and the two transmission signals are given to the transducer 11. In the transducer 11, ultrasonic waves which are obtained by the respective transmission signals are combined with each other, and the resulting synthesized ultrasonic wave is transmitted to and received from a test body. The reception signal of the ultrasonic wave reflected by one reflector is obtained as shown by a reception signal R_{b} in FIG. 3B. The waveform g₂ in FIG. 3B shows leakage of the transmission signals.

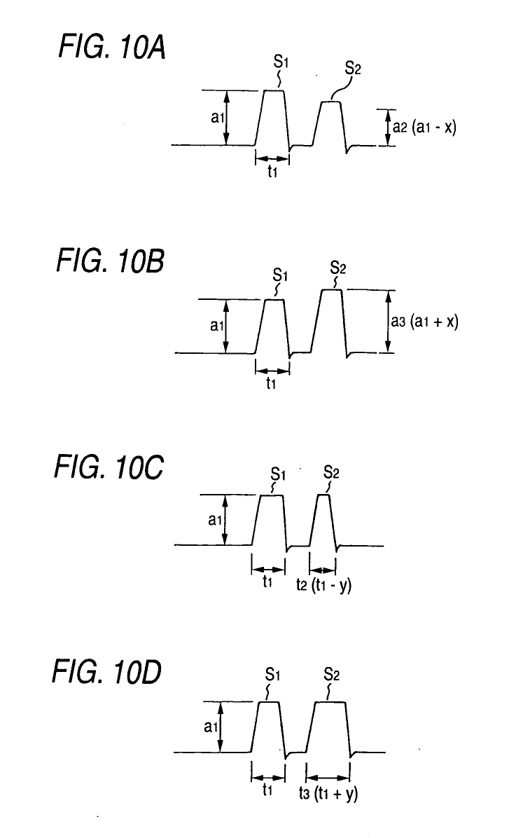

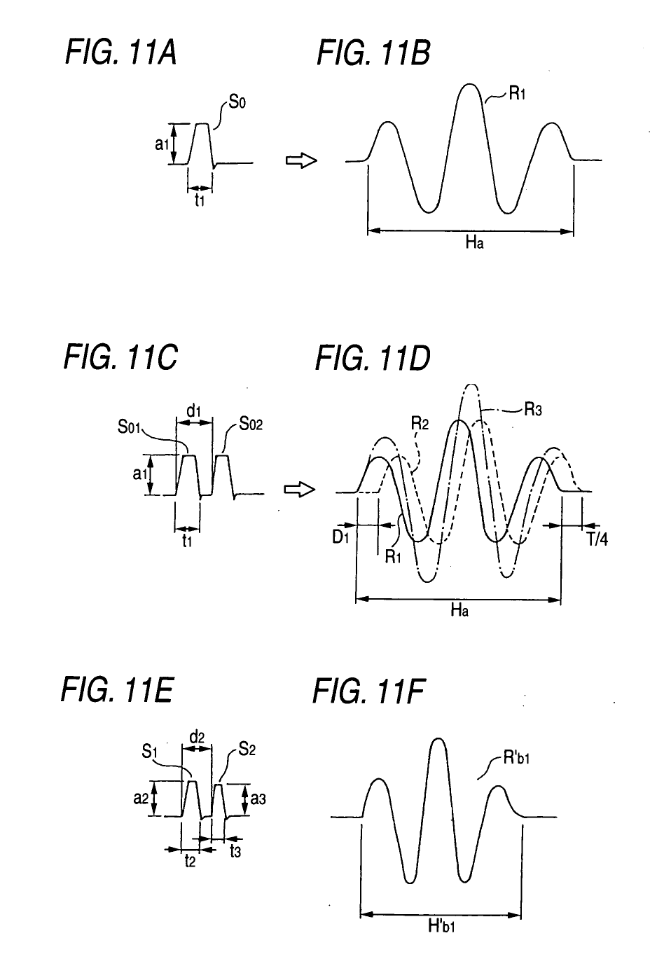

In the second embodiment, when the delay amount (time) d between the first transmission signal S₁ and the second transmission signal S₂ in FIG. 3A is variably adjusted, and the amplitudes and pulse widths of the transmission signals S₁, S₂ are variably adjusted as shown in FIGS. 10A to 10D, the distance resolution and the sensitivity can be enhanced as shown in FIGS. 11A to 11F to 13A to 13C. FIGS. 10A and 10B show examples in the case where the amplitude is changed. As shown in FIG. 10A, for example, a first transmission signal (pulse) S₁ having an amplitude a₁ and a pulse width t₁, and a second transmission signal (pulse) S₂ having an amplitude a₂ (=a₁−x) which is smaller than a₁, and the pulse width t₁ may be used, or, as shown in FIG. 10B, a first transmission signal S₁ having an amplitude a₁ and a pulse width t₁, and a second transmission signal S₂ having an amplitude a₃ (=a₁+x) which is larger than a₁, and the pulse width t₁ may be used.

FIGS. 10C and 10D show examples in the case where the pulse width is changed. As shown in FIG. 10C, for example, a first transmission signal S₁ having an amplitude a₁ and a pulse width t₁, and a second transmission signal S₂ having the amplitude a₁, and a pulse width t₂ (=t₁−y) which is shorter than t₁ may be used, or, as shown in FIG. 10D, a first transmission signal S₁ having an amplitude a₁ and a pulse width t₁, and a second transmission signal S₂ having the amplitude a₁, and a pulse width t₃ (=t₁+y) which is longer than t₁ may be used. In the first and second transmission signals S₁, S₂, alternatively, both the amplitude and the pulse width may be set to different values.

FIGS. 11A to 11F show waveforms in composing of ultrasonic waves for enhancing the distance resolution. In the case where a transmission signal S₀ of an amplitude a₁ and a pulse width t₁ is used as shown in FIG. 11A, an ultrasonic (reception) waveform due to the signal S₀ is obtained as a waveform R₁ in an ultrasonic wave generation period Hₐ shown in FIG. 11B. When the transmission signal S₀ is used as a first transmission signal S₀₁, and also as a second transmission signal S₀₂ with forming an interval of, for example, a previously adjusted delay amount d₁ as shown in FIG. 11C, the delay amount (time) D₁ of a second ultrasonic waveform R₂ with respect to a first ultrasonic waveform R₁ which is generated by the transducer 11 is T/4 (T: cycle of the ultrasonic waveform), and an ultrasonic wave (reception wave) of a waveform R₃ which is a combination of these ultrasonic waves (waveforms R₁ and R₂) is obtained as shown in FIG. 1D. The ultrasonic waveform R₃ is longer by a period of T/4 than the ultrasonic wave generation period Hₐ, but the wave height (amplitude) is higher than that in the case where an ultrasonic wave is generated by the single transmission signal S₀.

It will be seen that, when the amplitude (wave height) of the ultrasonic wave of FIG. 11D is reduced so as to be lowered to the level shown in FIG. 11B, the ultrasonic wave generation period H′_{b1} becomes shorter than the period Hₐ as indicated by the ultrasonic waveform R′_{b1} in FIG. 11F. In the embodiment, in order to obtain such an ultrasonic waveform, as shown in FIG. 11E, the first transmission signal S₁ having an amplitude a₂ (<a₁) and a pulse width t₂ (<t₁) which are smaller than those of the transmission signal S₀ of FIG. 11A, and a second transmission signal S₂ having an amplitude a₃ (<a₂) which is smaller than a₂, and a pulse width t₃ (<t₂) which is shorter than t₂ are used, whereby an ultrasonic wave [FIG. 11F] of the shorter ultrasonic wave generation period H′_{b1} is generated so that the distance resolution is enhanced. In FIG. 11F, while the period H′_{b1} is made shorter than that in the conventional art, the amplitude of the ultrasonic wave may be maintained higher than that in the conventional art. In this case, also the search sensitivity can be enhanced. Alternatively, the delay amount between ultrasonic waves may be set to nT/4 (n: an odd number), so that the ultrasonic wave generation period H′_{b1} can be efficiently shortened and the amplitude can be maintained high.

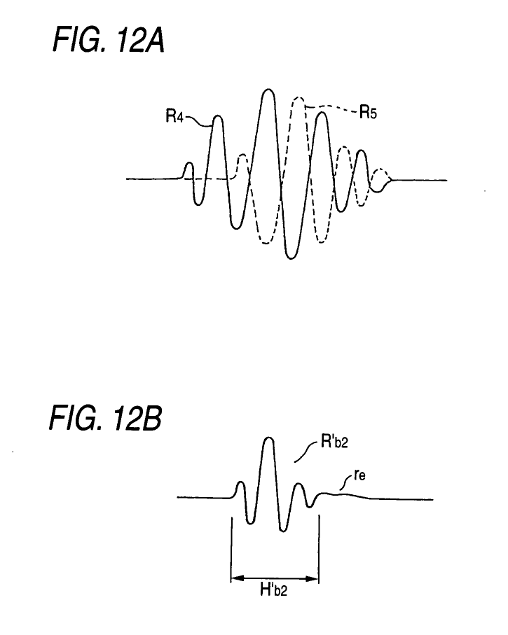

FIGS. 12A and 12B show another example of composing of ultrasonic waves for enhancing the distance resolution. When a first transmission signal S₁ such as shown in FIG. 11E and a second transmission signal S₂ which is different in amplitude and pulse width from S₁ are used, an ultrasonic waveform R₄ (solid line) and an ultrasonic waveform R₅ (broken line) can be combined with each other as shown in FIG. 12A. In this case, rear portions of R₄ and R₅ cancel each other, and a synthesized ultrasonic wave R′_{b2} of a short generation period H′_{b2} is obtained as shown in FIG. 12B. As indicated by rₑ in FIG. 12B, a small waveform may remain in the tail of the ultrasonic waveform. When the small waveform is low in level, however, the detection is not adversely affected.

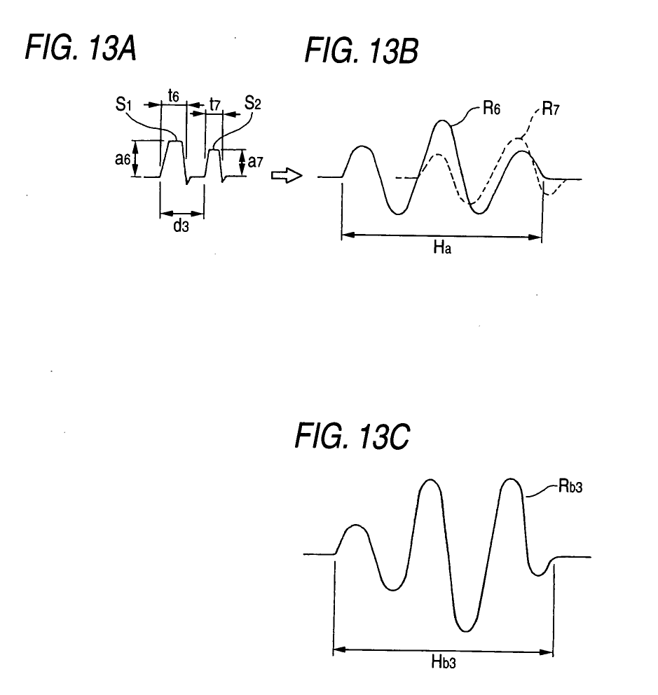

FIGS. 13A to 13C show waveforms in the case where the search sensitivity is to be enhanced. In this case, as shown in FIG. 13A, a first transmission signal S₁ having an amplitude a₆ and a pulse width t₆, and a second transmission signal S₂ having an amplitude a₇ (<a₆) which is smaller than a₆, and a pulse width t₇ (<t₆) which is shorter than t₆ are used in the same manner as FIGS. 11A to 11F, whereby an ultrasonic waveform R₇ (broken line) generated by the transmission signal S₂ is combined with an ultrasonic waveform R₆ (solid line) generated by the transmission signal S₁ with a delay amount of about one cycle as shown in FIG. 13B. Namely, the second transmission signal S₂ is output with forming a previously adjusted delay amount d₃ with respect to the first transmission signal S₁. Therefore, the ultrasonic waveforms R₆ and R₇ are combined with each other with the shift of about one cycle, and an ultrasonic waveform R_{b3} having a high amplitude is obtained as shown in FIG. 13C. The ultrasonic wave generation period H_{b3} of the ultrasonic waveform R_{b3} is slightly longer than the period Hₐ in the case where the combination is not conducted. However, the resulting ultrasonic waveform has a larger amplitude, and hence the search sensitivity can be enhanced.

In the above, the embodiment in which a synthesized ultrasonic wave is generated by the two transmission signals S₁, S₂ has been described. In order to further enhance the distance resolution or the sear sensitivity, an ultrasonic wave obtained by combination in which three or more successive transmission (pulse) signals are used may be transmitted and received in the same ultrasonic scan line (directions of transmitting and receiving waves). In the combining of ultrasonic waves, a small waveform may remain in the tail of each ultrasonic wave. When the small waveform is low in level, however, the detection is not adversely affected.

In the embodiment, in order to enhance the distance resolution or the sear sensitivity, the delay amount and number of the plural ultrasonic wave transmissions, and the amplitudes and pulse widths of the transmission signals are determined according to a probe identification code (the kind of the probe), or a selected or preset frequency of the ultrasonic wave, the transducer, and the like. In an ultrasonic diagnostic apparatus, various probes having different transducer characteristics are used, and there is a case where the frequency of an ultrasonic wave to be generated is selectable. In accordance with such situations, an optimum ultrasonic waveform must be obtained. In the first embodiment, therefore, information indicative of: the delay amount (d) for the second and subsequent transmission signals corresponding to the probe identification code or different ultrasonic wave frequencies; and the number of outputs of the transmission signals is stored and held. When the probe identification code of a connected probe is checked, or when the selected or preset frequency of the ultrasonic wave is checked, therefore, it is possible to control transmission and reception of an ultrasonic wave by the delay amounts and number of transmission signals corresponding to the identification code or the frequency of the ultrasonic wave. In the second embodiment, information indicative of: the delay amount (d) for the second and subsequent transmission signals corresponding to the probe identification code or different ultrasonic wave frequencies; and the number, amplitudes, and pulse widths of outputs of the transmission signals is stored and held. When the probe identification code of a connected probe is checked, or when the selected or preset frequency of the ultrasonic wave is checked, therefore, it is possible to control transmission and reception of an ultrasonic wave by the delay amounts, number, amplitudes, and pulse widths of transmission signals corresponding to the identification code or the frequency of the ultrasonic wave.

According to the ultrasonic diagnostic apparatus of the invention, the ultrasonic waveform is controlled with changing the delay amount for the second and subsequent ultrasonic waves with respect to the first ultrasonic wave, whereby the distance resolution and the search sensitivity can be enhanced, so that an image which has a high image quality, and which is easily observed can be obtained.

According to the ultrasonic diagnostic apparatus of the invention, plural transmission signals in each of which the output timing (delay amount) is adjusted are used in order to combine ultrasonic waves, and the amplitudes or pulse widths of the transmission signals are controlled to have different values, so that an arbitrary ultrasonic waveform is formed. As a result, the distance resolution and the search sensitivity can be enhanced, so that an image which has a high image quality, and which is easily observed can be obtained.

The entire disclosure of each and every foreign patent application from which the benefit of foreign priority has been claimed in the present application is incorporated herein by reference, as if fully set forth.

1. An ultrasonic diagnostic apparatus comprising a probe, the probe including at least one transducer that generates an ultrasonic wave, so as to form an ultrasonic tomographic image,
wherein a plurality of transmission signals are generated to at least one of said at least one transducer in a common scan line period, said plurality of transmission signals including a first transmission signal and at least one second transmission signal subsequent to the first transmission signal,
wherein said at least one second transmission signal is generated when a first ultrasonic wave resulting from the first transmission signal is generated, so as to form at least one second ultrasonic wave, and
wherein the first ultrasonic wave and said at least one second ultrasonic wave are combined with each other, so as to form a synthesized ultrasonic wave.

2. An ultrasonic diagnostic apparatus according to claim 1, wherein an initial one of said at least one second ultrasonic wave is generated with forming a shift of approximately nT/4 where n is an odd number and T is a cycle of a waveform of the first ultrasonic wave, to control a generation period of the synthesized ultrasonic wave to be shortened as compared with that of the first ultrasonic wave.

3. An ultrasonic diagnostic apparatus according to claim 1, wherein adjacent ones of a plurality of ultrasonic waves are generated with forming a shift of approximately one cycle, said plurality of ultrasonic waves resulted from said plurality of transmission signals, so as to control an amplitude of the synthesized ultrasonic wave to be increased as compared with that of the first ultrasonic wave.

4. An ultrasonic diagnostic apparatus according to claim 1, wherein an amplitude of each of said plurality of transmission signals is variably adjusted.

5. An ultrasonic diagnostic apparatus according to claim 4, wherein a pulse width of each of said plurality of transmission signals is variably adjusted.

6. An ultrasonic diagnostic apparatus according to claim 1, wherein a pulse width of each of said plurality of transmission signals is variably adjusted.

7. An ultrasonic diagnostic apparatus comprising:
a probe that includes a transducer generating an ultrasonic wave;
a transmitting section that outputs a plurality of transmission signals to the transducer;
a receiving section that receives a reception signal from the transducer;
a detection section that detects the reception signal, the detection section being connected to the receiving section;
an A/D converter that analog/digital-converts an output of the detection circuit;
a digital scan converter that applies scan conversion to an output of the A/D converter;
a monitor that displays an ultrasonic image based on an output of the digital scan converter; and
a control section connected to the transmitting section,
wherein the transmitting section comprises: at least one transmitting circuit; and a delaying circuit connected to at least one of said at least one transmitting circuit and to the control section,
wherein the control section sends a plurality of transmission triggers to said at least one transmitting circuit, at least one of said plurality of transmission triggers being routed through the delaying circuit,
wherein each of said at least one transmitting circuit receives each of said plurality of transmission triggers in the different timing, so as to output each of said plurality of transmission signals in the different timing.

8. An ultrasonic diagnostic apparatus according to claim 7, wherein the control section sends, to one of said at least one transmitting circuit, one of said plurality of transmission triggers which is not being routed through the delaying circuit, and sends, to the delaying circuit, the other one of said plurality of transmission triggers.

9. An ultrasonic diagnostic apparatus according to claim 7, further comprising an amplitude control section that variably adjusts an amplitude of each of said plurality of transmission signals.

10. An ultrasonic diagnostic apparatus according to claim 7, further comprising a pulse width control section that variably adjusts a pulse width of each of said plurality of transmission signals.
